(19) United States
(12) Patent Application Publication
(10) Pub. No.: US 2022/0338937 A1
(43) Pub. Date: Oct. 27, 2022

(54) Device For Navigating A Medical Instrument Relative To A Patient Anatomy

(71) Applicant: Stella Medical GbR, Munich (DE)

(72) Inventors: Laura Schütz, Munich (DE); Caroline Brendle, Munich (DE)

(73) Assignee: Stella Medical GbR, Munich (DE)

(21) Appl. No.: 17/763,493

(22) PCT Filed: Sep. 25, 2020
(86) PCT No.: PCT/EP2020/076880

§ 371 Date: Mar. 24, 2022

(30) Foreign Application Priority Data
Sep. 25, 2019 (DE) 10 2019 125 853.7

(51) Int. Cl. A61B 34/20; A61B 90/00

(52) U.S. Cl. CPC A61B 34/20; A61B 90/37; A61B 2034/2048; A61B 2034/2055; A61B 2034/2063

(57) ABSTRACT

The present invention relates to a device for navigating a medical instrument relative to a patient anatomy, a method for navigating a medical instrument relative to a patient anatomy, and a program element which, when executed by a computer, executes this method. The device comprises a position determination unit, a computing unit and a navigation display. The position determination unit comprises a sensor module configured to acquire current 3D data of the patient anatomy. The position determination unit further comprises a position sensor which is configured to acquire current movement data of the medical instrument. The computing unit is configured to match the current 3D data of the patient anatomy and the current movement data of the medical instrument with preoperative image data of the patient anatomy and, on this basis, to calculate navigation information for the medical instrument. The navigation display is configured to show the calculated navigation information.

### TECHNICAL FIELD

The present invention relates to a device for navigating a medical instrument relative to a patient anatomy, a method for navigating a medical instrument relative to a patient anatomy, and a program element which, when executed by a computer, executes the method.

### BACKGROUND OF THE INVENTION

Medical navigation appliances are nowadays used particularly in surgery, for example in neuronavigation or in orthopedic navigation of hip, knee, shoulder or spinal implants. For this purpose, marker elements and/or reference units are attached to the body of a patient and to surgical instruments or appliances. Sensor and/or camera modules set up in the room detect these marker elements and thus detect a position of the patient and of the instruments or appliances in the room. In this way, relative positions of the instruments, of the appliances and of the patient with respect to one another are detected, and their movements are determined in real time.

To permit real-time position determination and navigation display, conventional medical navigation systems require that a line of sight be constantly maintained between sensors and marker elements. However, this line of sight can restrict operating surgeons and operating personnel in their freedom of movement and can require them to adopt a specific, rigid arrangement of component parts of the navigation system into which they have to fit/adapt themselves. Moreover, the conventional navigation systems have large appliance footprints of the individual component parts and thus take up a lot of room in an already very limited floor space of an operating theater.

The use of today's medical navigation systems also entails radiation exposure for a patient and often also for an operating surgeon. The reason for this lies in the use of intraoperative X-ray, computed tomography or magnetic resonance tomography images, which are used for patient registration and/or intraoperative location and position determination of the instrument or implant.

A device for navigating a medical instrument relative to a patient anatomy can therefore be further improved.

### SUMMARY OF THE INVENTION

It is therefore an object of the present invention to make available a device which serves for navigating a medical instrument relative to a patient anatomy and which offers greater flexibility and user-friendliness in operation.

This object is achieved by a device for navigating a medical instrument relative to a patient anatomy and a method and a program element according to the independent claims. Advantageous embodiments and developments are set forth in the subclaims and in the following description.

The present invention comprises a device for navigating a medical instrument relative to a patient anatomy. The device comprises a position determination unit, a computing unit and a navigation display. The position determination unit comprises a sensor module which is configured to acquire current 3D data of the patient anatomy. The position determination unit further comprises a position sensor which is configured to acquire current movement data of the medical instrument. The computing unit is configured to match the current 3D data of the patient anatomy and the current movement data of the medical instrument with preoperative image data of the patient anatomy and, on this basis, to calculate navigation information for the medical instrument. The navigation display is configured to show the calculated navigation information.

The advantage of the device according to the invention for navigating a medical instrument relative to a patient anatomy is in particular that, during the operation, the patient and the operating surgeon are not exposed to any radiation due to intraoperative X-ray, computed tomography or magnetic resonance tomography recording. In addition, registration of the patient without use of marker elements or reference elements can be made possible by virtue of real-time recording and comparison of the three-dimensional patient anatomy with the preoperative image data.

The position determination unit can consist of at least two parts, namely the sensor module and the position sensor. The sensor module can comprise at least one sensor which is suitable for acquiring current or real-time 3D data of the patient anatomy, e.g. optical, magnetic field or ultrasonic sensors. However, the sensor module can preferably have a plurality of sensors, in order to increase the accuracy of the detection. The sensor module can thus, for example, have two optical sensors and a projection source, which together detect and create a three-dimensional surface structure of a patient anatomy. The position sensor can be a speed sensor or a location sensor that detects a current movement and/or orientation of the medical instrument.

The acquired sensor data of the patient anatomy and of the movement of the instrument can be sent to the computing unit, where they can be compared and matched with the preoperative patient data from, for example, ultrasound, X-ray, computed tomography or magnetic resonance tomography recordings. The computing unit can thereafter provide information on the position of the medical instrument in relation to the patient and can output patient data and navigation information via the navigation display.

In this way, the use of intraoperative X-ray, computed tomography or magnetic resonance tomography recordings can be avoided. By dispensing with a sensor module set up in the room, which usually detects marker elements, the device according to the invention can also enable a smaller footprint. Moreover, greater freedom of movement and faster set-up of a navigation system can be achieved by virtue of their being no need for calibration of marker elements and/or reference elements. This shortened set-up time and the omission of intraoperative image recordings can also save time in the operating theater and can allow hospitals to make more efficient use of operating theater capacity.

In one embodiment, the device further comprises the medical instrument, wherein the position determination unit, the computing unit and/or the navigation display are/is integrated in the medical instrument.

The medical instrument can be a portable instrument and comprise a surgical tool such as a drill, saw, milling cutter or an assisting instrument such as a guide rod, guide sleeve, working sleeve, trocar, endoscope or an implant such as a screw, rod, needle, thread, tissue or the like. By integrating the position determination unit, the computing unit and/or the navigation display in the medical instrument, it is possible to remedy both a line of sight problem caused by spatial separation between a sensor module and an instrument, and also the cognitive complexity for the operating surgeon caused by a spatial separation of the work region and a representation of the navigation information. In particular, the navigation display, which is integrated in the medical instrument, can serve as a main source for representation of navigation information relevant to the operation.

In this way, the device according to the invention can eliminate a spatial separation between navigation information and operating site. A user can therefore have all the relevant navigation information displayed directly in the operating field of view and can thus concentrate on the patient and on the work region (operating site). The navigation information can be processed in such a way that cognitive complexity is reduced to a minimum and the user can therefore focus primarily on manual execution of critical operating steps.

In addition, the device with an integrated position determination unit, computing unit and/or navigation display permits a compact set-up of a navigation system and thus affords freedom of movement and a free field of vision, particularly in the work region.

In another embodiment, the device comprises the medical instrument, but the position determination unit, the computing unit and/or the navigation display are/is releasably attached to the medical instrument with the aid of an adapter. The adapter can have a housing and a connection mechanism. At least one of position determination unit, computing unit and navigation display can be installed in the housing, and the housing can be mounted on the medical instrument via the connection mechanism. The connection mechanism can be, for example, a clamp connection, screw connection, plug connection or adhesive connection, as a result of which the housing can be releasably attached to the medical instrument.

The device for navigating a medical instrument relative to a patient anatomy can furthermore comprise an external computing unit and/or an external screen output, which are arranged at a distance from the computing unit and/or the navigation display. The current data acquired by the position determination unit can be transmitted to the external computing unit via a cable or via a wireless connection. The navigation information processed by the external computing unit can then be output via the external screen output or the navigation display mounted on the medical instrument.

The navigation display and the external screen output can be used together or individually. The transmission of the sensor data and navigation information between the position determination unit and the integrated computing unit or the external computing unit can take place either via a cable or via a wireless connection.

In one embodiment, the device for navigating a medical instrument relative to a patient anatomy can have marker elements in order to support the acquisition of the 3D data of the patient anatomy.

In one embodiment, the device for navigating a medical instrument relative to a patient anatomy can use intraoperative patient data for a registration process. Use of preoperative patient image data for localization of a medical instrument may be preferred because there is no radiation exposure. However, localization of a medical instrument on the basis of intraoperative patient image data may likewise be possible.

In one embodiment, the device comprises the medical instrument and has a cylindrical or rod-like shape with a rear end to be directed toward a user. The navigation display is arranged at the rear end of the device.

The cylindrical or rod-like medical instrument can have two end sections, wherein a first end section is directed toward the operating site and has a position determination unit. A second end section can be directed toward a user and have the navigation display, which is likewise directed toward the user. In this way, the user can receive the current navigation information in his operating field of view without constantly averting his eyes from the operating site.

In one embodiment, the medical instrument can have a pistol-like shape with a pistol grip and a pistol barrel, wherein the navigation display is arranged at the rear end of the pistol barrel. The pistol grip can be configured for better holding the instrument, and the navigation display can likewise be directed toward the user, in order to permit user-friendly presentation of the navigation information.

In one embodiment, the navigation display is configured to display an indication of the acquisition of the current 3D data of the patient anatomy, an indication of the accuracy of registration of the patient, an indication of the three-dimensional orientation and/or positioning of the medical instrument relative to the patient anatomy, and/or an indication of the working orientation and/or positioning of the medical instrument relative to the patient anatomy, and/or an indication of the work progress. Thus, the indications can comprise, for example, a status of a recording process of the patient anatomy, a patient registration, an operation plan, a site of use, an angle of use and/or a depth of use of the medical instrument, assistive instrument or implant.

In one embodiment, the indications are shown on the navigation display in the form of graphic patterns, preferably without image data of the patient anatomy. The navigation display can visualize the navigation indications of the medical instrument in the form of a pattern and a color. However, the indications do not have to contain any patient image data or patient information and can dispense with superpositioning of the image data. In particular, the indications can be shown on the navigation display in the form of at least one section of an arc of a circle and/or a crosshair and/or a target. However, the pattern can also be square or rectangular. Furthermore, the navigation display can, for example, use different colors to illustrate a target accuracy and/or a handling instruction.

In one embodiment, the device is portable and comprises the medical instrument, and the medical instrument is configured to be held in a human hand. Integrating or attaching the position determination unit, the computing unit and/or the navigation display to the medical instrument permits flexible and portable use of the device. By virtue of the compact set-up of the device, a large device footprint of the individual component parts of the device can also be avoided, such that a lot of room can be saved in an already very limited floor space of an operating theater. Furthermore, the portable device can be held in a user's hand or attached to a robotic arm in an automated operating environment.

In one embodiment, the navigation display is a projection which is projected by a projection unit onto the patient anatomy and/or onto a plane lying between an operating surgeon and a patient and/or displayed thereby. In this way, the navigation indications can be displayed directly on the patient anatomy and/or on a transparent surface. As an alternative, the navigation display can be a projection but also at the same time a display, on which the navigation indications are shown, or only a display.

The present invention further comprises a method for navigating a medical instrument relative to a patient anatomy, comprising the following steps:
acquiring current 3D data of the patient anatomy with the aid of a position determination unit,
acquiring current movement data of the medical instrument with the aid of the position determination unit,
matching the current 3D data of the patient anatomy and the current movement data of the medical instrument with preoperative data of the patient anatomy and, on this basis, calculating navigation information for the medical instrument with the aid of a computing unit, and
displaying the calculated navigation information with the aid of a navigation display.

In this way, any intraoperative radiation exposure for patient and operating surgeon can be avoided, and a registration of the patient can be made possible by real-time recording and comparison of the three-dimensional patient anatomy with the preoperative data.

Moreover, greater freedom of movement and faster set-up of a navigation system can be achieved. This shortened set-up time can also save time in the operating theater and allow hospitals to make more efficient use of operating theater capacity.

The present invention further comprises a program element which, when executed by a computer, executes the above-described method for navigating a medical instrument.

The present invention can be used for navigating a medical instrument relative to a living patient anatomy and also relative to an artificial anatomy simulation. The navigation system can also be suitable for use in a training environment.

Further features, advantages and possible uses of the present invention will emerge from the following description, the exemplary embodiments and the figures. All of the described and/or graphically illustrated features can be combined with one another irrespective of their illustration in individual claims, figures, sentences or paragraphs. In the figures, the same reference signs are used for identical or similar elements.

### BRIEF DESCRIPTION OF THE FIGURES

FIG. 1 shows a device for navigating a medical instrument relative to a patient anatomy, according to one embodiment of the invention.

FIGS. 2a-2d show a medical instrument with an integrated position determination unit, computing unit and navigation display, according to one embodiment of the invention.

FIGS. 3a-3c show representations of navigation information on a navigation display, according to one embodiment of the invention.

FIGS. 4a-4d show a medical instrument with a releasably attached position determination unit and navigation display, according to a further embodiment of the invention.

### DETAILED DESCRIPTION OF THE EXEMPLARY EMBODIMENTS

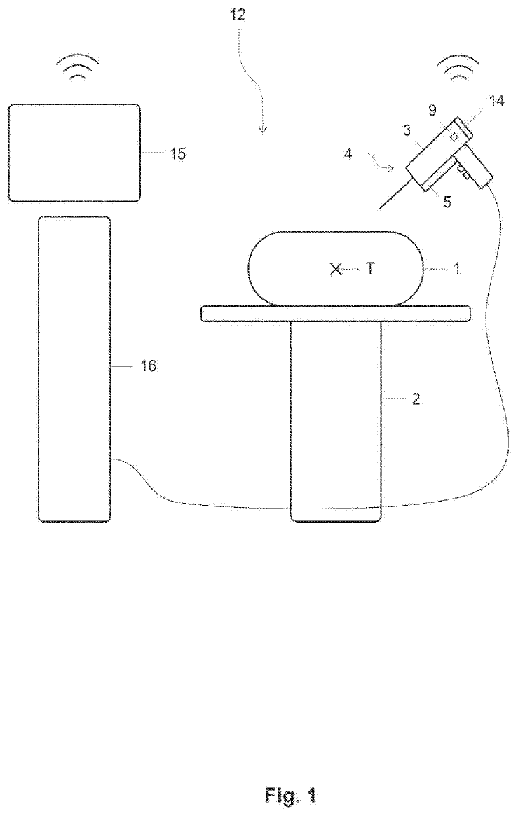

FIG. 1 shows a device 12 for navigating a medical instrument 3 relative to a patient anatomy 1. The medical instrument 3 can be portable and can be either a surgical tool, an assistive instrument or an implant. Thus, the medical instrument 3 can be held in a human hand or mounted on a robotic arm.

The device 12 comprises a position determination unit 4, a computing unit 16 and a navigation display 14. The position determination unit 4 comprises a sensor module 5 which is configured to acquire current 3D data of the patient anatomy 1. The sensor module 5 can comprise at least one sensor which is suitable for acquisition of current or real-time 3D data of the patient anatomy, e.g. optical, magnetic field or ultrasonic sensors.

The position determination unit 4 further comprises a position sensor 9 which is configured to acquire current movement data and/or alignment data of the medical instrument 3. The position sensor 9 can be a speed or location sensor, for example. The computing unit 16 is configured to match the current 3D data of the patient anatomy 1 and the current movement data of the medical instrument 3 with preoperative image data of the patient anatomy 1 and, on this basis, to calculate navigation information for the medical instrument 3, for example a site of use T.

The navigation display 14 is configured to display the calculated navigation information. Patient information, registration accuracy, operation planning data, preoperative patient image data, for example from ultrasound, X-ray, computed tomography or magnetic resonance tomography, and, if necessary, the navigation information can be displayed on an external screen output 15.

The user can interact with the device via an analog or digital input interface on the medical instrument 3, on the navigation display 14 or also on the external computing unit 16 or the external screen output 15.

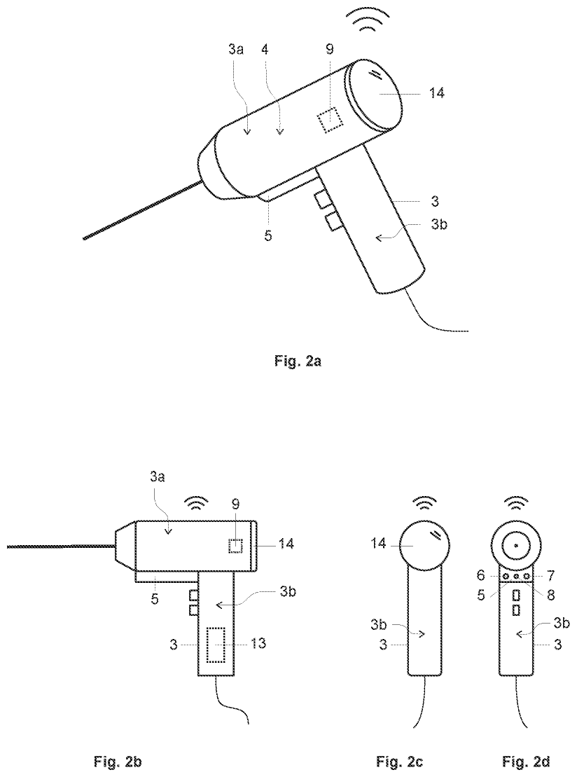

FIGS. 2a to 2d show a medical instrument 3 with an integrated position determination unit 4, computing unit 13 and navigation display 14, according to one embodiment of the invention. The medical instrument 3 can have a cylindrical or rod-like shape with a rear end 3a to be directed toward a user, or a pistol-like shape with a pistol barrel 3a and a pistol grip 3b.

The sensor module 5 is mounted on the pistol barrel 3a and comprises two optical sensors 6, 7 and a projection source 8, which are configured to create a three-dimensional surface structure of the patient anatomy. Furthermore, the position sensor 9 is integrated in the pistol barrel 3a, and the computing unit 13 is integrated in the pistol grip 3b. As an alternative, the position sensor 9 and the computing unit 13 can be integrated together in the pistol barrel 3a or in the pistol grip 3b.

On the basis of the received current 3D data of the patient anatomy 1 and the current movement data of the medical instrument 3, the computing unit 13 supplies an indication of the position of the medical instrument in relation to the patient. This navigation information can then be output via the navigation display 14, which is integrated at the rear end of the pistol barrel 3a to be directed toward the user.

The navigation information can comprise indications of the acquisition of the current 3D data of the patient anatomy 1, indications of the three-dimensional orientation and/or positioning of the medical instrument 3 relative to the patient anatomy 1, and/or indications of the work orientation and/or positioning of the medical instrument 3 relative to the patient anatomy 1, and/or indications of the work progress.

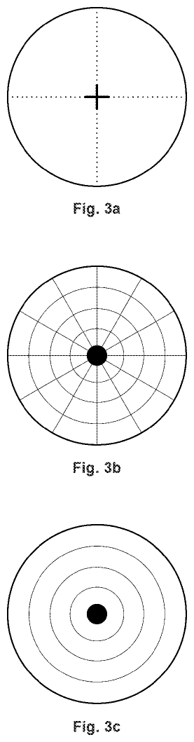

As is shown in FIGS. 3a to 3c, the indications can be viewed in the form of graphic patterns, preferably without image data of the patient anatomy, on the navigation display 14. In particular, FIG. 3a shows a representation of a site of use of the medical instrument 3, shown in the form of a crosshair. FIG. 3b shows a representation of an angle of use of the medical instrument 3, shown in the form of an arc of a circle. FIG. 3c shows a representation of a depth of use of the medical instrument 3, shown in the form of a target. Furthermore, the navigation display can, for example, use different colors to illustrate target accuracy and/or handling instructions.

In this way, it is possible to do without an external computing unit or an external screen output, with the result that the medical instrument 3, in addition to its original function, becomes a fully functional navigation system that is transportable and freely movable.

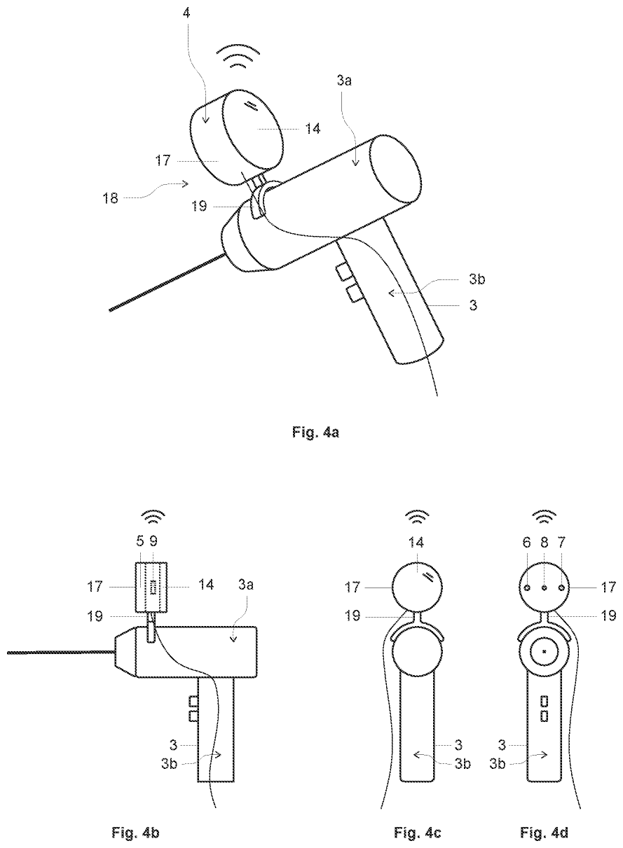

FIGS. 4a to 4d show a medical instrument 3 on which the position determination unit 4 and the navigation display 14 can be releasably attached with the aid of an adapter 18. The adapter 18 has a housing 17 and a connection mechanism 19. At least one of position determination unit 4, computing unit 13 and navigation display 14 can be arranged in the housing 17, and the housing 17 can be releasably attached to the medical instrument 3 with the aid of the connection mechanism 19, for example a clamp connection. Preferably, at least the position determination unit 4 and the navigation display 14 are arranged in the housing 17, with the navigation display 14 being able to be directed toward the user. The current sensor data acquired from the position determination unit 4 can be transmitted to the external computing unit 16 via a cable 10 or a wireless connection 11, e.g. WLAN or Bluetooth. The processed navigation information can be presented via the navigation display 14, optionally also the external screen output 15.

It should be noted that “comprising” and “having” do not exclude other elements or steps. It should further be noted that features or steps that have been described with reference to one of the above exemplary embodiments can also be used in combination with other features or steps of other exemplary embodiments described above. Reference signs in the claims are not to be regarded as restrictive.

1. A device for navigating a medical instrument relative to a patient anatomy, comprising:
a position determination unit,
a computing unit, and
a navigation display,
wherein the position determination unit comprises a sensor module which is configured to acquire current 3D data of the patient anatomy,
wherein the position determination unit further comprises a position sensor which is configured to acquire current movement data of the medical instrument,
wherein the computing unit is configured to match the current 3D data of the patient anatomy and the current movement data of the medical instrument with preoperative image data of the patient anatomy and, on this basis, to calculate navigation information for the medical instrument, and
wherein the navigation display is configured to show the calculated navigation information.

2. The device of claim 1, further comprising the medical instrument, wherein the position determination unit, the computing unit or the navigation display is integrated in the medical instrument.

3. The device as claimed in claim 1, further comprising the medical instrument, wherein the position determination unit, the computing unit or the navigation display is releasably attached to the medical instrument with the aid of an adapter.

4. The device as claimed in claim 1, wherein the device comprises the medical instrument and has a cylindrical or rod-like shape with a rear end to be directed toward a user, and the navigation display is arranged at the rear end of the device.

5. The device as claimed in claim 1, wherein the navigation display is configured to display an indication of the acquisition of the current 3D data of the patient anatomy, an indication of the three-dimensional orientation or positioning of the medical instrument relative to the patient anatomy, or an indication of the working orientation or positioning of the medical instrument relative to the patient anatomy, or an indication of the work progress.

6. The device as claimed in claim 1, wherein the indications are displayed on the navigation display (14) in the form of graphic patterns, preferably without image data of the patient anatomy (1).

7. The device as claimed in claim 1, wherein the device is portable and comprises the medical instrument, and the medical instrument is configured to be held in a human hand.

8. The device as claimed in claim 1, wherein the navigation display is a projection which is projected by a projection unit onto the patient anatomy or onto a plane lying between operating surgeon and patient or is displayed thereby.

9. A method for navigating a medical instrument relative to a patient anatomy, comprising the following steps:
acquiring current 3D data of the patient anatomy with the aid of a position determination unit,
acquiring current movement data of the medical instrument with the aid of the position determination unit,
matching the current 3D data of the patient anatomy and the current movement data of the medical instrument with preoperative data of the patient anatomy and, on this basis, calculating navigation information for the medical instrument with the aid of a computing unit, and
displaying the calculated navigation information with the aid of a navigation display.

10. A non-transitory program element which, when executed by a computer, executes the following steps for navigating a medical instrument relative to a patient anatomy, comprising:
acquiring current 3D data of the patient anatomy with the aid of a position determination unit,
acquiring current movement data of the medical instrument with the aid of the position determination unit,
matching the current 3D data of the patient anatomy and the current movement data of the medical instrument with preoperative data of the patient anatomy and, on this basis, calculating navigation information for the medical instrument with the aid of a computing unit, and
displaying the calculated navigation information with the aid of a navigation display.
